(19) United States
(12) Patent Application Publication
(10) Pub. No.: US 2010/0217991 A1
(43) Pub. Date: Aug. 26, 2010

(54) SURGERY ROBOT SYSTEM OF SERVER AND CLIENT TYPE

(76) Inventor: Seung Wook Choi, Gyeonggi-do (KR)

Correspondence Address: BIRCH STEWART KOLASCH & BIRCH, PO BOX 747, FALLS CHURCH, VA, 22040-0747, US

(21) Appl. No.: 12/680,647

(22) PCT Filed: Mar. 18, 2009
(86) PCT No.: PCT/KR09/01385

§ 371 Date: Mar. 29, 2010

(30) Foreign Application Priority Data
Aug. 14, 2008 (KR) 10-2008-0079821

(51) Int. Cl. H04L 9/32; H04L 9/00; G06F 17/30; A61B 19/00

(52) U.S. Cl. 713/176; 380/255; 726/15; 606/130; 340/5.81; 707/792; 707/E17.044

(57) ABSTRACT

A server-client type surgical robot system is disclosed. One aspect of the present invention provides a surgical robot system that includes a plurality of control clients, which generate control signals, and a surgical server, which is manipulated in correspondence with the control signals received from authenticated control clients. The server-client type surgical robot system can include a plurality of control clients for manipulating one surgical server, and incorporates security technology in server-client based robot surgery, to allow greater safety in performing surgery.

### CROSS-REFERENCE TO RELATED APPLICATIONS

This application claims foreign priority benefits under 35 U.S.C. 119(a)-(d) to PCT/KR2009/001385, filed Mar. 18, 2009, which is hereby incorporated by reference in its entirety.

### BACKGROUND

The present invention relates to a surgical robot system, more particularly to a server-client type surgical robot system.

In the field of medicine, surgery refers to a procedure in which a medical device is used to make a cut or an incision in or otherwise manipulate a patient's skin, mucosa, or other tissue, to treat a pathological condition. One type of surgery, a laparotomy, is a surgical procedure in which the skin of the abdomen is cut open and an internal organ, etc., is treated, reconstructed, or excised.

Especially when conducting a laparotomy, a portion of skin may be cut and lifted upwards to form a particular amount of space between the skin and the tissue, and the surgical operation may be performed within this space. As a laparotomy may cause many scars and may thus entail a lengthy healing period, laparoscopic surgery has recently been proposed as an alternative. Laparoscopic surgery generally involves making a small incision in the abdomen of the patient and performing surgery while observing the surgical site within the abdominal cavity using a laparoscope inserted through the incision. Laparoscopic surgery is also widely used in various fields of medicine, including surgeries such as cholecystectomies, appendectomies, gastrectomies, proctocolectomies, etc., as well as urology, gynecology, and obstetrics. The laparoscope is an apparatus used for the imaging diagnosis of an internal organ and typically includes a miniature camera. The laparoscope may be inserted into the body, and the image information retrieved by the miniature camera may be observed through an external monitor.

Also, in situations where it is difficult to approach the patient, such as in combat zones, spacecraft, and in the absence of professional clinical staff, a current method of performing remote surgery may be employed, using a remote surgery system. This method is to have a doctor perform surgery by remotely manipulating a master robot, using a communication system, to control a slave robot that is positioned close to the patient. A conventional surgical robot may be based on the master-slave concept, in which a maneuver on the master side is copied exactly on the slave side. However, in cases where one robot needs to be maneuvered by several doctors, for example when a doctor at the location of the surgery and a specialist at a remote location are to conduct a surgical procedure together, a plurality of robots may be needed for inputting manipulations, and the master-slave concept may not be an appropriate choice.

The information in the background art described above was obtained by the inventors for the purpose of developing the present invention or was obtained during the process of developing the present invention. As such, it is to be appreciated that this information did not necessarily belong to the public domain before the patent filing date of the present invention.

### SUMMARY

An aspect of the present invention is to provide a surgical robot system that includes a plurality of control clients for manipulating one surgical server.

Another aspect of the present invention is to provide a surgical robot system that incorporates security technology in server-client based robot surgery and thereby allows greater safety in performing surgery.

Other technical problems addressed by the present invention will be readily understood from the descriptions that follow.

One aspect of the present invention provides a surgical robot system that includes a plurality of control clients, which generate control signals, and a surgical server, which is manipulated in correspondence with the control signals received from authenticated control clients.

A surgical robot system based on an embodiment of the present invention may further include a security server that is configured to receive an identifier from the plurality of control clients and perform authentication.

The plurality of control clients can include: a first control client that is configured to transmit an instrument control signal, which is for controlling a surgical instrument included on the surgical server, to the surgical server; and a second control client that is configured to transmit a vision control signal, which is for controlling a vision system included on the surgical server, to the surgical server.

Also, the security server may provide a different type of authority for each of the plurality of control clients.

The control signals can be transmitted to the surgical server in an encrypted state, and can be transmitted to the surgical server via a virtual private network (VPN).

The security server may authenticate the control clients by a digital signature scheme. A server-client type surgical robot system according to an embodiment of the present invention can further include a storage unit for storing information on the history of access to the surgical server by the plurality of control clients.

Here, the history information can include one or more types of information selected from a group consisting of identifiers, access times, session information, and type of operation of the plurality of control clients, and a combination thereof.

Also, the control client can include a caller unit, configured to generate a set of call information and transmit the call information to another control client, where the call information may include one or more type of information selected from a group consisting of text information, image information, voice information, sound information, and a combination thereof.

A server-client type surgical robot system according to an embodiment of the present invention can also include a control right designator unit, which may designate a particular control client from among the plurality of control clients to manipulate the surgical server.

The server-client type surgical robot system according to an aspect of the present invention can include a plurality of control clients for manipulating one surgical server, and incorporates security technology in server-client based robot surgery, to allow greater safety in performing surgery.

Additional aspects, features, and advantages, other than those described above, will be obvious from the claims and written description below.

### BRIEF DESCRIPTION OF THE DRAWINGS

FIG. 1 represents the structure of a server-client type surgical robot system according to an embodiment of the present invention.

FIG. 2 illustrates a control client in a server-client type surgical robot system according to an embodiment of the present invention.

FIG. 3 is a block diagram representing a security server in a server-client type surgical robot system according to an embodiment of the present invention.

### DETAILED DESCRIPTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention.

While terms including ordinal numbers, such as “first” and “second,” etc., may be used to describe various components, such components are not limited to the above terms. The above terms are used only to distinguish one component from another.

When a component is said to be “connected to” or “accessing” another component, it is to be appreciated that the two components can be directly connected to or directly accessing each other but can also include one or more other components in-between.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Also, in providing descriptions referring to the accompanying drawings, those components that are the same or are in correspondence are rendered the same reference numeral regardless of the figure number, and redundant descriptions are omitted. In the written description, certain detailed explanations of related art are omitted, when it is deemed that they may unnecessarily obscure the essence of the present invention.

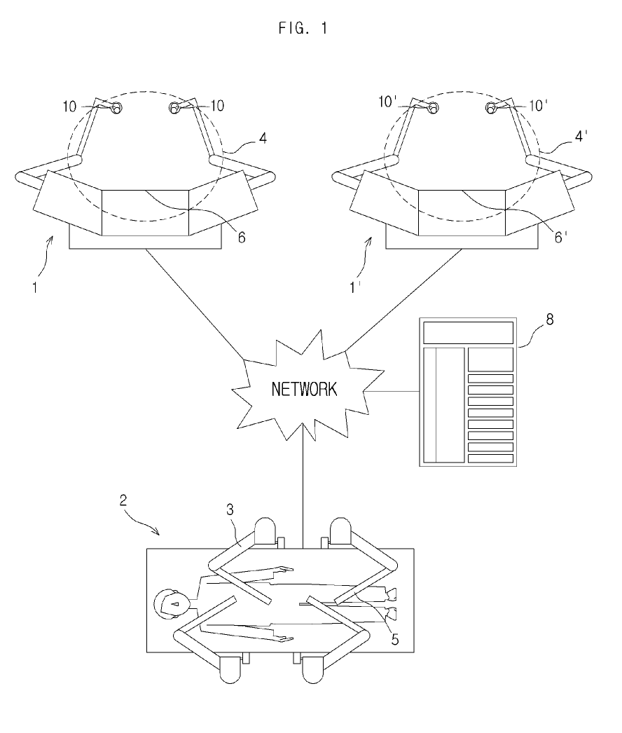

FIG. 1 represents the structure of a server-client type surgical robot system according to an embodiment of the present invention. Illustrated in FIG. 1 are control clients 1, 1′, a surgical server 2, robot arms 3, control interfaces 4, 4′, a laparoscope 5, monitors 6, 6′, a security server 8, and handles 10, 10′. According to this embodiment, a vision system for providing images during surgery, for example a system for controlling equipment such as a laparoscope, an endoscope, a microscope, a magnifier, a reflector, etc., can be coupled to the surgical server 2. The following descriptions will be provided using an example in which the vision system is a system for controlling a laparoscope.

A feature of this embodiment is to include a plurality of control clients 1, 1′ to manipulate a surgical server 2, which actually performs surgery on the patient, so that various numbers of control clients 1, 1′ may be used in manipulating the surgical server 2 depending on factors such as the difficulty of the surgical procedure, the location of the surgical site, the participation of medical specialists, etc. While there are just two control clients 1, 1′ illustrated, it is obvious that the control clients 1, 1′ can be included in greater numbers.

In this embodiment, the control interface 4, 4′ is a concept that encompasses not only the manipulation handles mounted on the surgical control client 1, 1′, but also the processors for signal processing, consoles, monitors 6, 6′, and other operating switches connected to the handles. The control interface 4, 4′ serves as an interface which identifies user manipulation on the control client 1, 1′ to operate the surgical server 2.

A user conducting surgery may manipulate the handles 10, 10′ provided on a control client 1, 1′, to manipulate the surgical server 2. The handles 10, 10′ can be connected to the control client 1, 1′, which may be manipulated directly by the user. The control client 1, 1′ may remotely control the robot arms 3 and laparoscope coupled to the surgical server 2, to perform surgery by having the robot arms 3 and laparoscope move and rotate in space, make incisions in or capture images of the surgical site, and so on.

The user may move and rotate the robot arms 3 and make incisions, etc., by holding the handles 10, 10′ with one hand or both hands and using the buttons attached to the handles 10, 10′. A laparoscope 5 can be inserted to visually observe the surgical site during surgery. The laparoscope 5 may be inserted close to the surgical site where the robot arms 3 are inserted.

The handles 10, 10′ can be implemented in various mechanical structures according to the manipulation method and can include various inputting means, such as a keypad, trackball, touchscreen, etc., for operating the robot arms 3 and other surgical equipment.

The user can manipulate the handles 10, 10′ while watching an image of the inside of the operating room in which the surgical server 2 is located through a display unit 6, 6′. A camera (not shown) for showing the operating room may selectively photograph important scenes within the operating room. Also, the display units 6, 6′ may output an image of the inside of the abdominal cavity, as photographed by the laparoscope. The display units 6, 6′ can output a plurality of images. In this case, the area of the display units 6, 6′ can be implemented by hardware or software. An arrangement including more than one monitor can output the image information for one area on each of the monitors, while an arrangement including one monitor can output different types of information by dividing the display into several windows.

The control clients 1, 1′ may be coupled by a wired or a wireless network to the surgical server 2 located at the site of the surgery. The control clients 1, 1′ and the surgical server 2 may include transceivers for network communication and can form a server-client network. That is, the surgical server 2 can be the server that deals with the network operations, while the control clients 1, 1′ can manipulate the surgical server 2 by transmitting control signals for controlling certain devices, such as the surgical instruments, laparoscope, robot arms 3, etc., to the surgical server 2. The control signals can be generated by the manipulation on the handles 10, 10′. On each of the robot arms 3, a surgical instrument, laparoscope, suction equipment, irrigation equipment, etc., can be mounted.

A plurality of control clients 1, 1′ can control the single surgical server 2 at the same time. For example, a first control client 1 may perform surgery by maneuvering a surgical instrument coupled to the surgical server 2, and a second control client 1′ may maneuver laparoscope coupled to the surgical server 2, while another control client may be manipulated by an assistant to perform a procedure such as suction, irrigation, etc., using a tool coupled to the surgical server 2. For this purpose, the first control client 1 may transmit an instrument control signal to the surgical server 2 for controlling a surgical instrument, and the second control client 1′ may transmit a vision control signal (for instance, a laparoscope control signal) to the surgical server 2 for controlling a vision system (for instance, a laparoscope). The surgical server 2 may receive the control signals and manipulate the coupled devices, e.g. the surgical instrument and the laparoscope, in correspondence to the signals.

Also, according to this embodiment, certain control clients 1, 1′ can be designated to have control rights to the surgical server 2 according to the professionalism of the user. For example, one control client 1 can maneuver the surgical server 2 to perform surgery at the site of the surgical procedure, and at another point in time when a difficult procedure is required, the control right to the surgical server 2 may be transferred to another control client 1′.

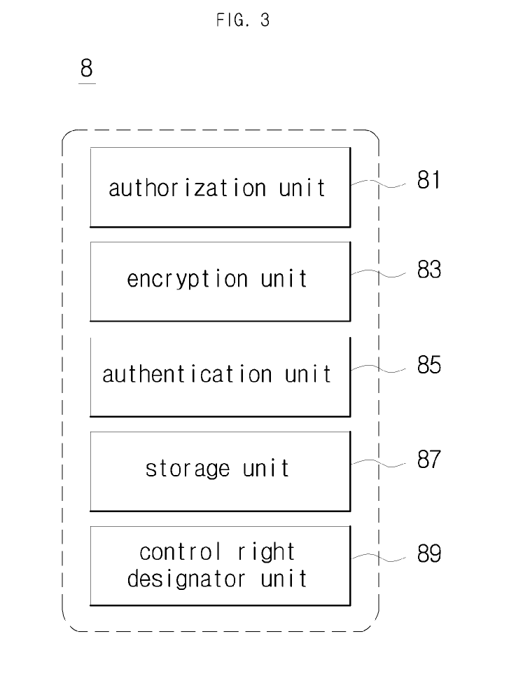

The security server 8 can be a server provided for applying security technology to the communication between the plurality of control clients 1, 1′ and the surgical server 2. The security server 8 can be implemented as a module within the surgical server 2 or as a separate device. The security server 8 can be coupled in parallel or in serial to the control clients 1, 1′ and the surgical server 2. Since many control clients 1, 1′ may access a single surgical server 2, it may be required to verify which one is attempting access and with what authority. To satisfy these requirements, security technology may be used to provide functions such as identification, authentication, authorization, confidentiality, integrity, and audit trail, for example. This will be described later in further detail with reference to FIG. 3.

The control right to the surgical server 2 can be transferred between the plurality of control clients 1, 1′ in various ways. The control rights of the control clients 1, 1′ can be designated according to the authorization of the security server 8. According to an embodiment of the present invention, one control client 1 can transfer a control right designation signal for a particular function to the surgical server 2, after which the security server 8 can determine whether or not the control client 1 has the authority for the function in question and determine whether or not to provide authorization. If the control client 1 is authorized, the control client 1 can control the surgical server 2 for the particular function. The control right designation signal can be a signal requesting that the control client 1 that generated the signal be designated the control right to the surgical server 2 or a signal requesting that another control client be designated the control right to the surgical server 2. The latter case may be used when one doctor manipulating a control client 1 requests another doctor manipulating a different control client 1′ to proceed with the surgery.

The control right designation signal can be divided according to function. For example, a control client 1 capable of performing various functions, such as manipulating a surgical instrument, manipulating a laparoscope, manipulating a suction device, and manipulating an irrigation device, may request a control right designation by transferring a control signal designation signal for each function to the surgical server 2. The security server 8 may determine whether or not the control client 1 has the authority for each of the functions and respond to the control right designation request accordingly, so that the control client 1 may control the surgical server 2. The security server 8 may include pre-stored information in a storage unit on the authorities provided to each of the control clients 1, 1′, and when there is a request for a control right designation, the security server 8 may designate the control right to a control client 1, 1′ using information stored in the storage unit regarding control capabilities for each function.

According to another embodiment of the present invention, a user may personally designate a certain control client 1, 1′ for performing a particular function, by using a surgical console (not shown) coupled to the surgical server 2. The surgical console (not shown) can also be one of the control clients 1, 1′. For example, the control right to the surgical server 2 belonging to a first control client 1 can be allotted to a second control client 1′ by a user manipulating a surgical console, which is coupled to the surgical server 2 and which oversees the overall surgical procedure. With this system, a user overseeing the overall surgery can personally change the control client that conducts a surgical procedure, according to a need for specialist techniques or for stepwise surgical operations. In this case, the control right to the surgical server 2 of the first control client 1 can remain intact or can be cancelled. Whether or not to maintain the control right may be determined according to the designation of the user on the surgical console.

For this allotment of control rights, the surgical console can include a control right designator unit. The control designator unit can allot the control right to each device coupled to the surgical server 2 for each of the control clients. For example, the control right designator unit may allot the control right to a surgical instrument to a first control client 1 and allot the control right to a laparoscope to a second control client 1′. Information regarding this allotment of control rights can be stored in a database and can be modified by a user through a user interface (UI). Here, the user interface may include buttons and a screen (including a touchscreen) to enable the user to allot the control rights to certain devices to certain control clients.

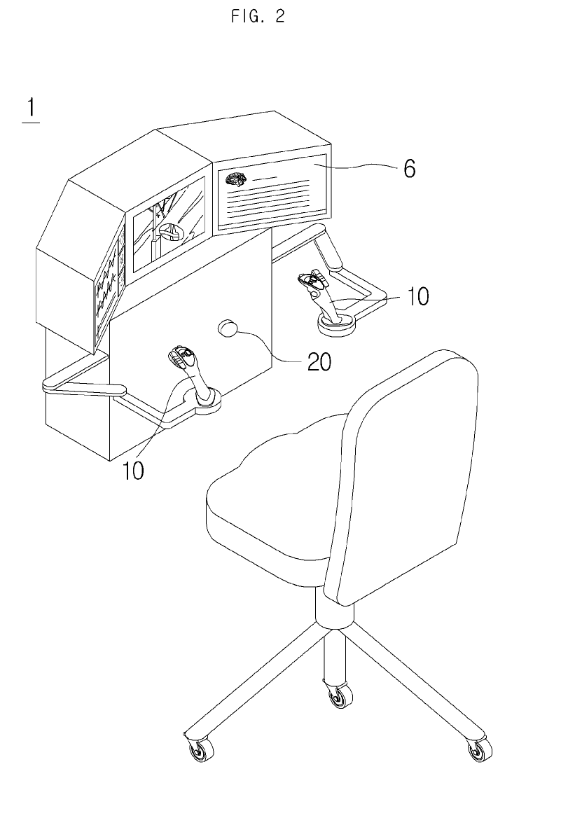

FIG. 2 is an illustration of a first control client 1 in a server-client type surgical robot system according to an embodiment of the present invention. Referring to FIG. 2, the first control client 1 can include a display unit 6, handles 10, and a caller unit 20. The following descriptions will focus on differences from the previously described embodiment.

The caller unit 20 may generate a set of call information and transmit it to a second control client 1′. The call information can be information exchanged between control clients 1, 1′ or information for calling each other. For example, when a doctor manipulating the first control client 1 wishes to communicate with another doctor manipulating the second control client 1′ or request designation of control rights to the surgical server 2, the doctor may transmit the necessary information by using the caller unit 20.

The set of call information can include one or more types of information selected from a group consisting text information, image information, voice information, sound information, and a combination of the above. If the call information is text information, for instance, the caller unit 20 can be a text-inputting device (e.g. a device for inputting preselected phrases or a keyboard, etc.), and the text information can be outputted on the display unit 6′ of the second control client 1′.

If the call information is voice information, the caller unit 20 can be a microphone, and the inputted voice information can be outputted from a speaker (not shown) included in the second control client 1′. A speaker can also be included in the first control client 1 to allow the users of the control clients 1, 1′ to communicate with each other. By thus using the caller units 20, the users of the control clients 1, 1′ can communication with each other and request designation, etc.

FIG. 3 is a block diagram representing a security server 8 in a server-client type surgical robot system according to an embodiment of the present invention. Illustrated in FIG. 3 are an authorization unit 81, an encryption unit 83, an authentication unit 85, a storage unit 87, and a control right designator unit 89. For convenience, the following descriptions will be provided for an example in which the security server 8 is a separate device. It is obvious, however, that the following descriptions can also be applied to those cases where the security server 8 is implemented as a module in the surgical server 2.

The security server 8 may perform functions such as identification, authentication, authorization, confidentiality, integrity, and audit trail.

Identification refers to a process of checking “who” (which user) is accessing the system, using an identifier, such as a user ID, fingerprint scan, iris scan, for example. When a control client 1, 1′ transmits an identifier to the security server 8, the authentication unit 85 may determine whether or not the control client 1, 1′ is one that is allowed access to the system using the identifier. Since the identifier of the control clients 1, 1′ represents the identity of each device, it can also be used when analyzing the accountability of the user.

Authentication refers to a process of checking whether or not the accessed user is “real.” Methods of authentication include methods that are based on the knowledge of the user (e.g. passwords), methods using authentication devices owned by the user (e.g. keys, smart cards, tokens, etc.), methods that use the physical authentication characteristics of the user (e.g. fingerprint scans, voice recognition, iris scans, etc.), methods that utilize actions that are not consciously made by the user (e.g. digital signatures), and so on. If there are several control clients 1, 1′ in the hospital or in a remote location, a node authentication function can also be included, which is to check whether or not the user is attempting access from a specified device, in order to control which control client 1, 1′ a user gains access from. For this purpose, the authentication unit 85 may store an identifier (e.g. a protocol) for the node in question in the storage unit 87, and perform authentication using the node identifier for the device gaining access. Here, the control clients 1, 1′ and the security server 8 may mutually verify authentication using digital certificates.

Authorization refers to a process of checking the authority of a control client 1, 1′ that has gained access: whether the control client 1, 1′ has the authority to conduct surgery or is allowed to move only the laparoscope, etc. For this purpose, the authorization unit 81 may include pre-stored information in the storage unit 87 regarding the authorities for each function and each control client 1, 1′. The information on which user can access the system and to what extent the authority is granted (regarding the permitted access times and the types of devices that can be controlled, for example) can be predefined, so that when a user logs on to the computer operating system or to an application program, the authorization unit 81 may help the system or application to decide which resources the user will be allowed to use during this particular session. The granting of authority may be a process of verifying the authorities pre-designated by the authorization unit 81 and the authorities designated when the control clients 1, 1′ gain access.

Confidentiality is related to the technology of encrypting the communication between the control clients 1, 1′ and the surgical server 2, so that the communication may not be revealed to others. A variety of encryption techniques typically used in communication can be applied to this embodiment, and in addition, a virtual private network (VPN) can be established between control clients 1, 1′ and the surgical server 2. The encryption unit 83 may be pre-designated with the encryption method and may transmit information according to the encryption method or decode information received.

Integrity refers to a process for guaranteeing integrity in the communication between the control clients 1, 1′ and the surgical server 2. A technology such digital signatures can be applied to this embodiment. That is, for a control client that gains access by a digital signature method, the authentication unit 85 can provide authentication using the identifier and a digital certificate. Here, the digital certificate can be based on a public key infrastructure (PKI).

Audit trail refers to a process of managing all actions of the control clients 1, 1′ and surgical server 2 in the form of logs. The storage unit 87 can store information on the history of accesses to the surgical server 2 by the plurality of control clients 1, 1′. Here, the logs can be stored unaltered, or can be transmitted to a separate log server. The logs can be stored in the form of simple text files or XML documents, or in another form. The history information can include one or more types of information selected from a group consisting of identifiers, access times, session information, and type of operation of the plurality of control clients 1, 1′, and a combination the above.

The security server 8 can additionally include a control right designator unit 89, which may designate a particular control client from among the plurality of control clients 1, 1′ to manipulate the surgical server 2. In cases where a user directly specifies the control client 1, 1′ that will perform a particular function, as described above, the user can designate the control rights of a particular function to a particular control client 1, 1′ via the control right designator unit 89. In this case, the control right designator unit 89 can serve as a tool, such as an interface and a source editor, for modifying the control rights stored in the storage unit 87 in designating the control rights.

The description of other details related to the server-client type surgical robot system according to an embodiment of the present invention, including, for example, common platform technology, such as the embedded system, O/S, etc., interface standardization technology, such as the communication protocol, I/O interface, etc., and component standardization technology, such as for actuators, batteries, cameras, sensors, etc., will be omitted.

In the foregoing, the server-client type surgical robot system according to an aspect of the present invention has been disclosed for one particular embodiment, with regards the name, number, structure, and connection relationships of the control client 1, 1′, surgical server 2, and security server 8. However, the present invention is not limited to the disclosed embodiment, and an arrangement that includes different names, numbers, structures, and connection relationships can still be encompassed by the scope of the present invention, as long as the overall processes and effects are the same. As such, it is to be appreciated that various changes and modifications can be made by those skilled in the art without departing from the spirit and scope of the present invention as defined by the appended claims.

What is claimed is:

1. A surgical robot system comprising:
a plurality of control clients configured to generate a control signal; and
a surgical server manipulated in correspondence with the control signal received from authenticated control clients.

2. The surgical robot system of claim 1, further comprising:
a security server configured to receive an identifier from the plurality of control clients and perform authentication.

3. The surgical robot system of claim 1, wherein the plurality of control clients include:
a first control client configured to transmit to the surgical server an instrument control signal for controlling a surgical instrument included on the surgical server; and
a second control client configured to transmit to the surgical server a vision control signal for controlling a vision system included on the surgical server.

4. The surgical robot system of claim 1, wherein the security server provides a different authority for each of the plurality of control clients.

5. The surgical robot system of claim 1, wherein the control signal is transmitted to the surgical server in an encrypted state.

6. The surgical robot system of claim 1, wherein the control signal is transmitted to the surgical server via a virtual private network (VPN).

7. The surgical robot system of claim 1, wherein the security server authenticates the control clients by a digital signature scheme.

8. The surgical robot system of claim 1, further comprising:
a storage unit configured to store information on a history of access to the surgical server by the plurality of control clients.

9. The surgical robot system of claim 8, wherein the history information includes one or more types of information selected from a group consisting of identifiers, access times, session information, and type of operation of the plurality of control clients, and a combination thereof.

10. The surgical robot system of claim 1, wherein the control client comprises a caller unit configured to generate a set of call information and transmit the call information to another control client.

11. The surgical robot system of claim 10, wherein the call information includes one or more type of information selected from a group consisting of text information, image information, voice information, sound information, and a combination thereof.

12. The surgical robot system of claim 1, further comprising:
a control right designator unit configured to designate a particular control client from among the plurality of control clients to manipulate the surgical server.
